(19) United States
(12) Patent Application Publication
(10) Pub. No.: US 2006/0121137 A1
(43) Pub. Date: Jun. 8, 2006

(54) Muscadine grape pomace in the treatment of intestinal inflammation

(76) Inventors: Diane K. Hartle, Athens, GA (US); Phillip Greenspan, Athens, GA (US); James L. Hargrove, Athens, GA (US); Charles L. Hofacre, Watkinsville, GA (US); Eve E. Bralley, Athens, GA (US)

Correspondence Address: ICE MILLER LLP, ONE AMERICAN SQUARE, SUITE 3100, INDIANAPOLIS, IN, 46282-0200, US

(21) Appl. No.: 11/296,815

(22) Filed: Dec. 7, 2005

Related U.S. Application Data
(60) Provisional application No. 60/633,820, filed on Dec. 7, 2004.

(51) Int. Cl. A61K 36/87

(52) U.S. Cl. 424/766

(57) ABSTRACT

This application relates to a composition and method for treating or preventing inflammation. One embodiment of the composition comprises a dried powdered grape pomace. Further one embodiment of the methods of treating inflammation involves administering a muscadine grape product to an animal, and wherein the muscadine grape product is included in the diet or feed of the animal as an additive or supplement.

### PRIORITY

This application claims priority to U.S. Provisional Patent Application 60/633,820, filed Dec. 7, 2004, which is hereby incorporated by reference in its entirety.

### BACKGROUND

Enteritis is an inflammation of the intestine, which often causes severe cramping, diarrhea, and bloody stool. If left unchecked, enteritis may result in decreased absorption of nutrients by the intestine, ulceration or perforation of the intestine, and weakening of the mucus defenses of the intestines. There are many causes to enteritis, including bacterial, viral, protozoal, fungal, and autoimmune disfunctions which may cause such an inflammation. Indeed, enteritis is a common medical problem in humans, and an economically noteworthy problem in the poultry and animal husbandry industries. For example, the occurrence of enteritis in humans may cause dehydration or malnourishment, which is of particular concern in infants or small children. Further, enteritis in livestock, including poultry, has caused substantial economic loss dating back to at least 1961.

For example, necrotic enteritis has been a worldwide problem in the poultry industry since its first description in England in 1961. Most often associated with the bacterium Clostridium perfringens and/or protozoa of the genus Coccidia, necrotic enteritis is characterized by the necrosis (death) of the intestinal lining, often resulting in death of the infected animal. The poultry industry has noted that an outbreak of necrotic enteritis is often not discovered until sudden death of the animal, and an acute outbreak in poultry often claims 10% of the livestock. Further, in the event of low grade enteritis in the livestock, weight gain is hindered, thereby increasing the time and cost to bring livestock to market.

The industry response to combating enteritis and the resultant economic loss in livestock has been to inoculate the livestock's feed with low grade antibiotics, even when the livestock show no signs of enteritis. This practice has been dubbed “growth-promoting antibiotics,” and has come under increasing attack from a variety of agencies, advocacy groups, biomedical organizations, and citizen lobbies. In particular, opposition to the use of growth-promoting antibiotics has led to a ban of this practice in Europe and has caused a great deal of pressure to provide meat that is not produced with the aid of growth-promoting antibiotics.

Further, other inflammations regularly occur in animals and humans, including colitis, and the medical industry has seen a revival of interest in treatments that do not involve the use of traditional drugs or pharmaceuticals, when appropriate. Therefore, a composition and method for preventing or treating inflammation, including enteritis and colitis, that does not require the use of growth-promoting antibiotics or other traditional pharmaceuticals would be greatly appreciated.

### SUMMARY

The present application relates generally to a method and composition for the treatment of inflammation.

In particular, according to one embodiment, a composition for treating inflammation is considered. Specifically, a composition for the treatment of inflammation includes a therapeutic amount of dried powdered whole muscadine grape pomace. Optionally, the composition may be packaged for oral or rectal administration. Another option includes the provision of the whole muscadine grape pomace as a poultry feed additive or an additive in poultry feed.

The composition described above may, in addition to dried powdered whole muscadine grape pomace, include poultry feed such that the poultry feed comprises more than 90% by weight of the entire composition, and the grape pomace comprises less than 10% by weight of the composition. Other formulations including poultry feed exist. For example, one formulation may include less than 5% by weight grape pomace. Another formulation may include between about 0.5% to about 2.0% by weight grape pomace.

Yet another embodiment relates to a method for treating inflammation, including the administration of a muscadine grape product to an animal. The animal to which the muscadine grape product is administered may be, for example, avian or mammalian, and may further be human. Among other things, the inflammation treated may be intestinal inflammation such as enteritis or colitis. Further, among other things, the muscadine grape product administered to the animal may include whole muscadine grape pomace, grape skins, grape pomace, seed extract, seed extract, or a combination of those items. In one embodiment, the muscadine grape product is administered by causing the animal to ingest the muscadine grape product. Ingestion may be caused by providing the muscadine grape product as an additive to livestock feed. Alternately, another method of administering a muscadine grape product to an animal may be performed through colonic application, such as through the use of an enema.

Another embodiment involves a method of treating intestinal inflammation in an animal including the step of administering a composition having a fruit product high in phytochemicals. The fruit product may optionally be a grape product, and may include, among other things powdered dried skins, powdered dried pomace, and/or grape extract. The grape may be a muscadine grape. Further, the animal may be a vertebrate, including mammals, bird, and other vertebrates. The animal may be a human.

### BRIEF DESCRIPTION OF THE DRAWINGS

FIG. 1A is a photographic representation of the colon of a healthy animal.

FIG. 1B is a photographic representation of the colon of an animal displaying colitis traits after being treated with TNBS.

FIG. 1C is a photographic representation of the colon of an animal after being treated with TNBS to induce colitis, and with the animal ingesting a diet including muscadine grape pomace.

FIG. 1D is a photographic representation of the colon of an animal after being treated with TNBS to induce colitis, with the animal ingesting a diet including muscadine grape pomace, and having an enema including muscadine extract.

FIG. 2 is a chart representing the amount of Myeloperoxidase (MPO) activity at seven days for animals with various treatments, and showing the reduced MPO activity for those animals with TNBS-induced colitis receiving muscadine grape products.

### DESCRIPTION

The present application relates to compositions and methods for reducing or preventing inflammation in an animal, and for preventing or treating enteritis in an animal.

Experimentation by applicants, as summarized in Provisional Application No. 60/633,820 (hereby incorporated in its entirety by reference), has shown the biological activity of the muscadine grape (Vitis rotundifolia), and has shown muscadines to have one of the highest ORAC values, a measure of antioxidant potential, of any fruit. This grape is rich in polyphenolic and flavonoid compounds, and is high in other phytochemicals. In addition to the muscadine grape, other grapes, such as the table grape (Vinis vinifera), display similar properties and can be used in the place of the muscadine grape. Other fruits high in phytochemicals may also be expected to have similar results. However, the particularly high biological activity of the muscadine grape, as well as the various other characteristics outlined above, makes the muscadine grape a preferred fruit for treatment of inflammation.

According to one aspect of the present application, muscadine grape products are used to ameliorate the severity of acute inflammation in animals. Muscadine grape products may take the form of whole grapes, grape skins, grape pomace, seed extract, and/or grape extract. Further, muscadine grape products may be dried, frozen, freeze-dried, and/or powdered or flaked. In one exemplary embodiment, muscadine grape products are ingested by an animal such as a mammal or bird to treat against acute or chronic inflammation. In particular, whole powdered muscadine grape pomace was used as a feed additive for treating intestinal inflammation. For example, the muscadine grape product may constitute less than about 10% by weight of the total diet ingested by the animal.

In one exemplary embodiment, addition of between about 2% and 0.5% (by weight) whole muscadine pomace to chicken feed has been shown to diminish the degree of necrotic enteritis in chickens infected with Clostridium-perfringens and Coccidia. Results have indicated that that such a treatment results in infected chickens having an approximately 8% greater weight gain and an approximately 11%-15% reduced mortality rate compared to infected chickens not receiving muscadine pomace.

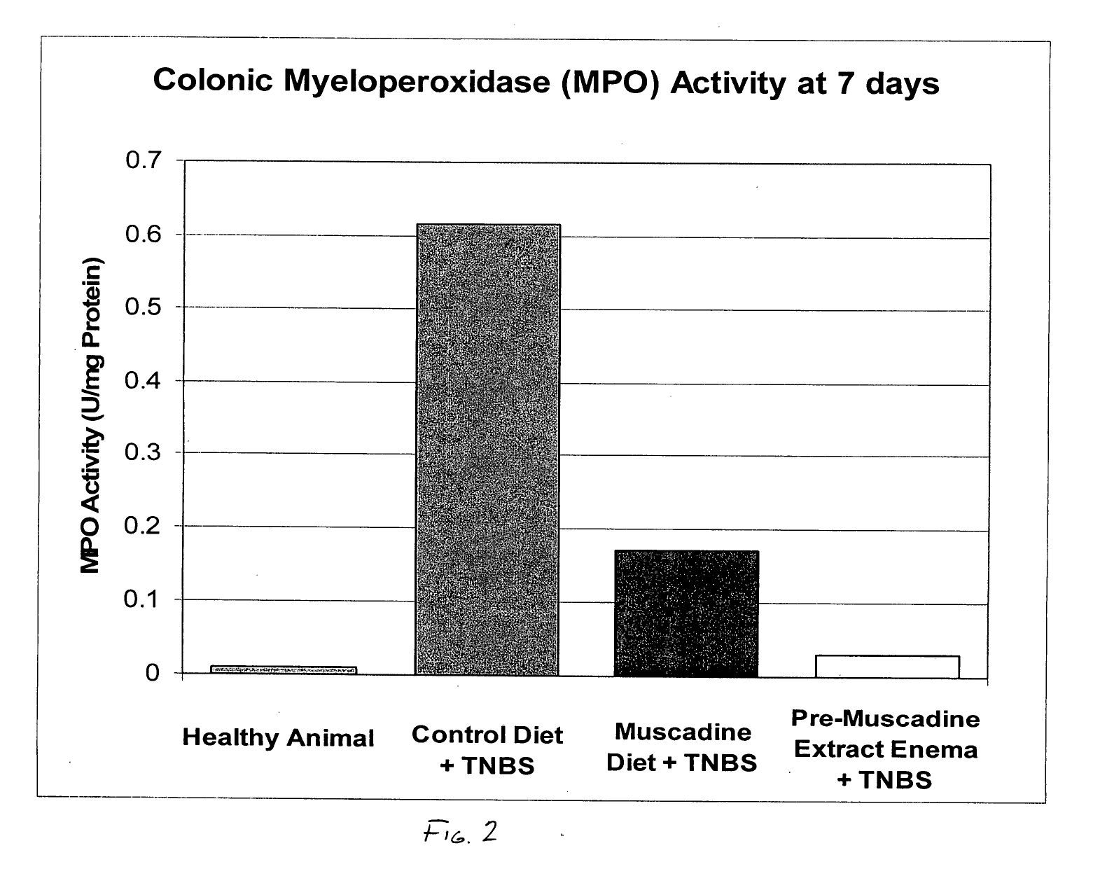

In another exemplary embodiment, addition of approximately 5% (by weight) of muscadine grape skin powder to the feed and approximately 5% (by volume) of muscadine grape skin powder to the water of animals has been shown to reduce and/or prevent colon inflammation in animals consuming the treated feed and water. For example, TNBS (2,4,6-trinitrobenzene sulfonic acid) was used to induce colitis in experimental animals to reproduce macroscopic, histological and immunological hallmarks of human ulcerative colitis by binding to proteins initiating immune activation, infiltration, inflammation, edema and severe colon ulceration. The experimental animals were fed the feed and water containing muscadine grape skin powder for three days prior to use of TNBS, which was administered intracolonically. As shown in FIG. 2, those experimental animals receiving 5% (by weight) of muscadine grape skin powder to the feed and approximately 5% (by volume) of muscadine grape skin powder to the water of animals showed a decrease in myeloperoxidase activities (“MPO,” a marker of neutrophil invasion and inflammation) by 75% over the control group, a decrease in edema of 40%, and a decrease in macroscopic scores when compared to a control group. Therefore, according to this example, it is apparent that muscadine phytochemicals exert a protective effect over inflammation, and may be used to prevent and treat inflammation.

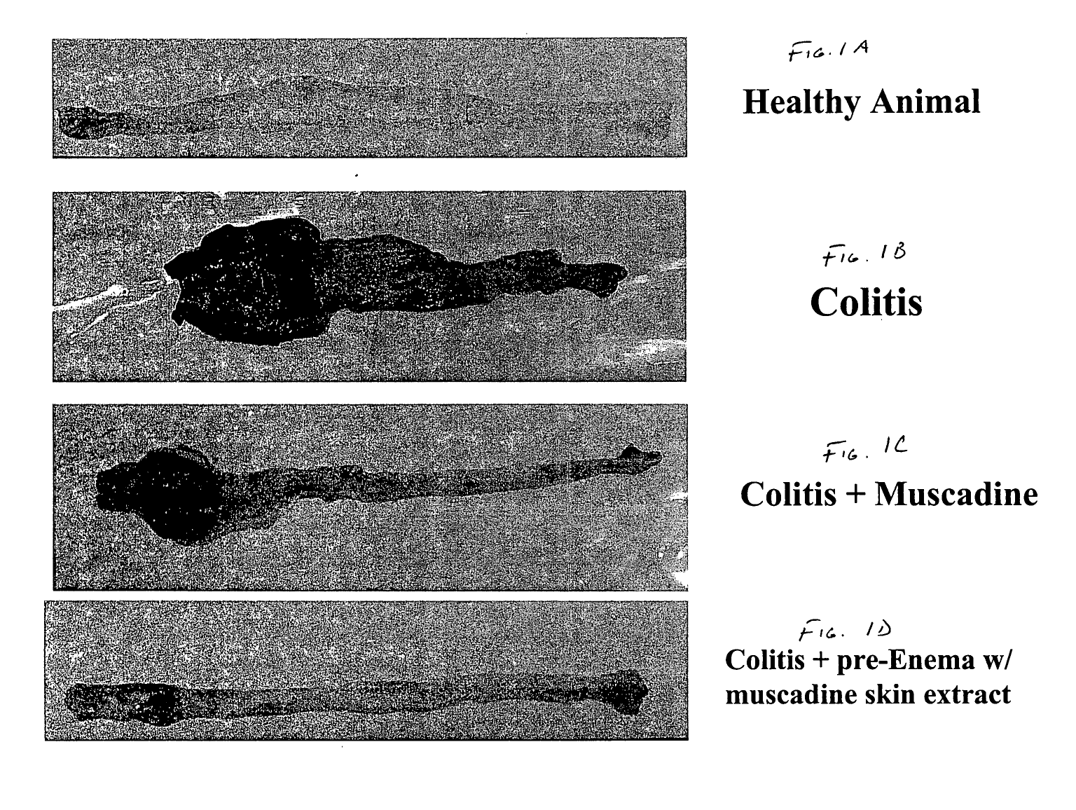

Yet another exemplary embodiment involves the use of approximately 5% (by weight) of muscadine grape skin powder to the feed and approximately 5% (by volume) of muscadine grape skin powder to the water of animals, and were administered with a muscadine extract enema 30 minutes after having TNBS administered intracolonically. As seen in FIGS. 1A-D and FIG. 2, those experimental animals receiving this treatment showed little to no inflammation in the colon, and MPO activity at seven days near that of a healthy animal.

Notably, the muscadine pomace feed additive performed as well or better than the industrial standard antibiotic (BMD). There was significant improvement in feed conversion and body weight gains and a decreased mortality rate in chickens fed the pomace. Therefore, it is evident from the experimental results obtained within these studies that a therapeutic amount of muscadine grape product may be less than 10% of the diet of an animal, and may further be less than 5% of the diet of an animal. According to one aspect of the present invention, as little as 0.5% of the diet of the animal may be a therapeutically effective amount of muscadine grape product in treatment of inflammation when administered orally.

Although the present invention has been described in considerable detail with reference to certain preferred versions thereof, other versions are possible. For example the applicability of the use of other fruits high in phytochemicals with a spectrum similar to muscadine grapes could be used. Additionally, experimentation in both poultry and rats have shown the efficacy of a therapeutic dosage of muscadine grape products in reducing inflammation. The experimental data showing effectiveness in two separate genera with consistent results indicates that this treatment and composition is effective in other animals as well. For example, the present composition and methods may be applied to vertebrates including mammals, birds, reptiles, fish, and amphibian. Further, according to another embodiment of the present invention, a therapeutically effective dose of the compositions described herein may also be calculated on the basis of percentage of caloric intake, with the muscadine grape product not exceeding more than 10% of the caloric intake of the animal. Therefore, the spirit and scope of the appended claims should not be limited to the description of the preferred versions contained herein.

What is claimed is:

1. A composition for the treatment of inflammation comprising a therapeutic amount of dried powdered whole muscadine grape pomace.

2. The composition of claim 1, wherein the composition is packaged for oral or rectal administration.

3. The composition of claim 1, wherein the dried powdered whole muscadine grape pomace is provided as an additive to poultry feed.

4. The composition of claim 1, further comprising poultry feed, wherein the dried powdered whole muscadine grape pomace comprises less than 10% by weight of the composition.

5. The composition of claim 1, further comprising poultry feed, wherein the dried powdered whole muscadine grape pomace comprises less than 5% by weight of the composition.

6. The composition of claim 1, further comprising poultry feed, wherein the dried powdered whole muscadine grape pomace comprises about 0.5% to about 2.0% by weight of the composition.

7. A method of treating inflammation comprising administering a muscadine grape product to an animal.

8. The method of claim 7, wherein the animal is avian or mammalian.

9. The method of claim 8, wherein the animal is a human.

10. The method of claim 7, wherein the inflammation is intestinal inflammation.

11. The method of claim 10, wherein the intestinal inflammation is due to enteritis or colitis.

12. The method of claim 7, wherein the muscadine grape product is selected from a group consisting of grape skins, grape pomace, seed extract, and grape extract.

13. The method of claim 7, wherein the muscadine grape product is administered by causing the animal to ingest the muscadine grape product.

14. The method of claim 13, wherein the muscadine grape product is provided as an additive to livestock feed.

15. The method of claim 14, wherein the muscadine grape product comprises less than 10% of the livestock feed.

16. The method of claim 7, wherein the muscadine grape product is administered colonically.

17. A method of treating intestinal inflammation in an animal comprising the step of administering a composition comprising a fruit product high in phytochemicals.

18. The method of claim 17, wherein the fruit product is a grape product.

19. The method of claim 18, wherein the grape product is selected from the group consisting of powdered dried skins, powdered dried pomace, and grape extract.

20. The method of claim 19, wherein the grape product is a muscadine grape product.

21. The method of claim 18, wherein the animal is a vertebrate.

22. The method of claim 21, wherein the vertebrate is a human.

23. The method of claim 21, wherein the vertebrate is avian.

24. The method of claim 17, wherein the method is directed to preventing inflammation.
